(19) United States
(12) Patent Application Publication
(10) Pub. No.: US 2024/0173463 A1
(43) Pub. Date: May 30, 2024

(54) PORTABLE HEMODIALYSIS SYSTEMS

(71) Applicant: University of Washington, Seattle, WA (US)

(72) Inventors: Barry Fulkerson, Seattle, WA (US); Jeremy Barribeau, Seattle, WA (US); Anna Galperin, Seattle, WA (US)

(73) Assignee: University of Washington, Seattle, WA (US)

(21) Appl. No.: 18/551,832

(22) PCT Filed: Mar. 22, 2022
(86) PCT No.: PCT/US2022/021401

§ 371 Date: Sep. 21, 2023

Related U.S. Application Data
(60) Provisional application No. 63/165,098, filed on Mar. 23, 2021.

(51) Int. Cl. A61M 1/16

(52) U.S. Cl. CPC A61M 1/1696

(57) ABSTRACT

Portable hemodialysis systems include a dialysis fluid module configured to regenerate a dialysis fluid, and a blood handling module reversibly fluidically couplable to the dialysis fluid module. The portable hemodialysis system is configured to dialyze blood in the blood handling module using the dialysis fluid regenerated by the dialysis fluid module.

### CROSS REFERENCE TO RELATED APPLICATIONS

This application claims the benefit of U.S. provisional patent application No. 63/165,098, filed Mar. 23, 2021, which is herein incorporated by reference.

### BACKGROUND

Healthy kidneys keep blood urea levels at a low, relatively constant value. While hemodialysis is an effective treatment to extend the lives of patients experiencing kidney failure, there are many known problems with hemodialysis systems and treatment methods. For example, patients undergoing maintenance dialysis (several times per week) show elevated urea levels that decline rapidly to very low urea levels in a typical dialysis session. This non-physiologic imbalance accounts for many of the discomforts experienced by patients between and after dialysis sessions, as well as longer-term complications. Additionally, known hemodialysis systems require connection to a water source and use 400 liters or more of water per dialysis session, a key barrier to wearable dialysis.

Known dialysis systems reliant upon single-use components create large amounts of medical waste and require very large volumes of water. Sorbent columns used with most wearable concepts to remove urea also lead to large amounts of medical waste, are expensive, heavy, and often generate toxic products.

Conventional hemodialysis achieves the removal of excessive metabolic waste from the body by running about 120 liters of dialysate per session, which typically requires 3-4 hours of treatment. The dialysis may be required three times a week. Patients are subjected to significant life disruptions, including having to be immobilized for hours and having to arrange transportation to dialysis centers, which impact their quality of life.

Therefore, a portable hemodialysis system that allows a patient to be ambulatory during dialysis would improve the patient's quality of life.

### BRIEF DESCRIPTION OF THE SEVERAL VIEWS OF THE DRAWINGS

The foregoing aspects and many of the attendant advantages of the claimed subject matter will become more readily appreciated by reference to the following detailed description, when taken in conjunction with the accompanying drawings, wherein:

FIG. 1 shows a partially hidden perspective view of a hemodialysis system according to an embodiment of the present disclosure.

FIG. 2 shows a hemodialysis system configured to fit in a wearable article, according to an embodiment of the present disclosure.

FIG. 3A shows an upper rear exploded perspective view of a hemodialysis system according to an embodiment of the present disclosure.

FIG. 3B shows an upper front exploded perspective view of the hemodialysis system of FIG. 3A.

FIG. 4 shows a perspective view of aspects of a dialysis module according to an embodiment of the present disclosure.

FIG. 5A shows a first perspective view of aspects of a dialysis fluid module and a dialysis module according to an embodiment of the present disclosure.

FIG. 5B shows a second perspective view of aspects of the dialysis fluid module and the dialysis module of FIG. 5A.

FIG. 5C shows a perspective view of aspects of a portion of dialysis fluid regeneration module according to an embodiment of the present disclosure.

FIG. 6 is an exploded view of a photo-oxidation module according to an embodiment of the present disclosure.

### DETAILED DESCRIPTION

Hemodialysis systems generally include a dialyzer unit which removes urea from a patient's bloodstream. In particular, a bloodstream is pumped through the dialyzer unit and separated from a flow of dialysate fluid (e.g., dialysate) by a membrane, which allows mass exchange for select molecules between the flow of blood and the flow of dialysis fluid. The partially cleaned bloodstream continues either back to the patient's vascular system or downstream to another module for further processing. The cleansed bloodstream is routed back to the patient's vascular system, resulting in a volume of “spent” dialysis fluid containing the urea and uremic toxins removed from the bloodstream.

Known hemodialysis systems typically require a connection to an external high-volume water source and use hundreds liters or more of water per dialysis session, a key barrier to wearable dialysis.

The present disclosure provides hemodialysis systems that greatly improve quality of life for dialysis patients by removing the required connection to an external water source, and by dramatically reducing the volumes of dialysis fluid required per treatment. In particular, the hemodialysis systems disclosed herein have a small, portable size and run continually on a small volume of recirculating dialysis fluid without the need for an external water connection—freeing patients to dialyze on the go, restoring their mobility during treatment and providing longer, more continuous dialysis that more closely mimics native kidney function.

Representative embodiments are described herein. Accordingly, alike term have alike meanings unless stated differently. The present disclosure expressly encompasses additional embodiments comprising combinations of features, and it shall be understood that any feature(s) of any embodiment may be combined with any other embodiment.

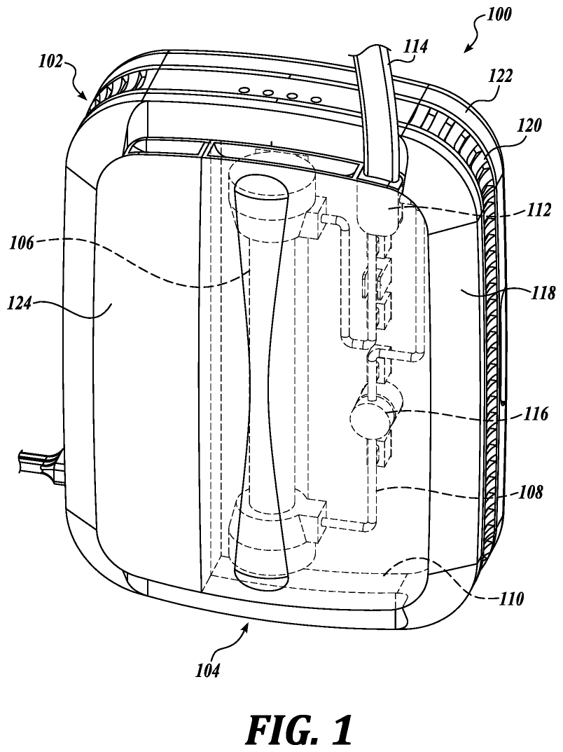

FIG. 1 shows a portable hemodialysis system 100 according to an embodiment of the present disclosure. The hemodialysis system 100 includes a dialysis fluid module 102 configured to regenerate a dialysis fluid by removing urea and uremic toxins from the dialysis fluid, and to pump such regenerated dialysis fluid through a dialysis fluid circuit. The hemodialysis system 100 also includes a blood handling module 104 reversibly fluidically couplable (i.e., dockable) to the dialysis fluid module 102, and in particular, to the dialysis fluid circuit. The blood handling module 104 includes a dialyzing membrane which separates a patient's bloodstream from dialysate fluid received from the dialysis fluid module via the dialysis fluid circuit, allowing mass exchange for select molecules between the bloodstream and the flow of dialysis fluid, thereby removing urea and uremic toxins (e.g., indoxyl sulfate) from the bloodstream.

Advantageously, the hemodialysis system 100 dialyzes blood in the consumable blood handling module 104 using dialysis fluid which is regenerated by the dialysis fluid module 102. Because the hemodialysis system 100 continuously regenerates dialysis fluid, the patient is freed from traditional, stationary hemodialysis systems connected to large dialysis fluid supplies.

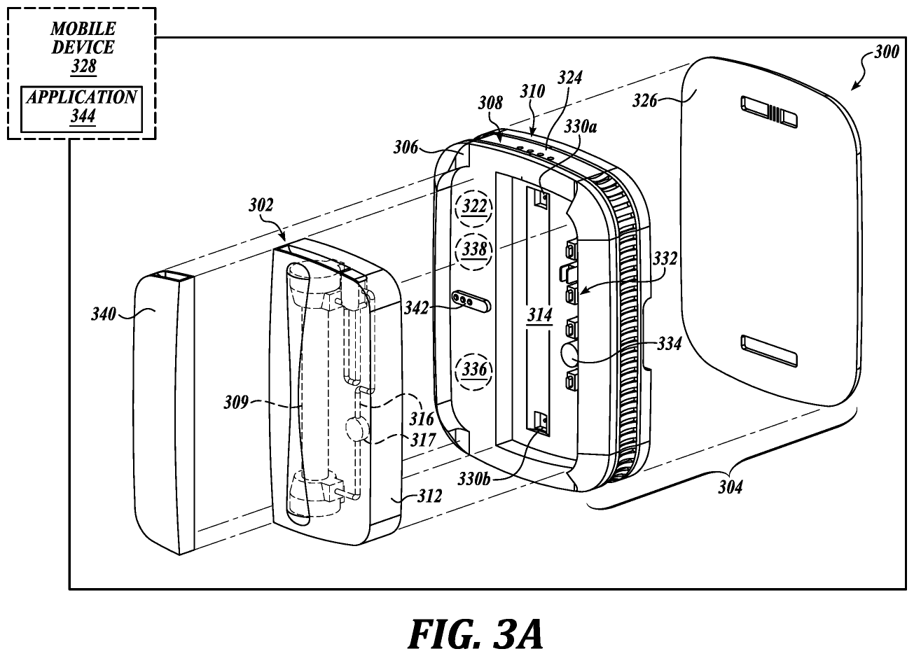

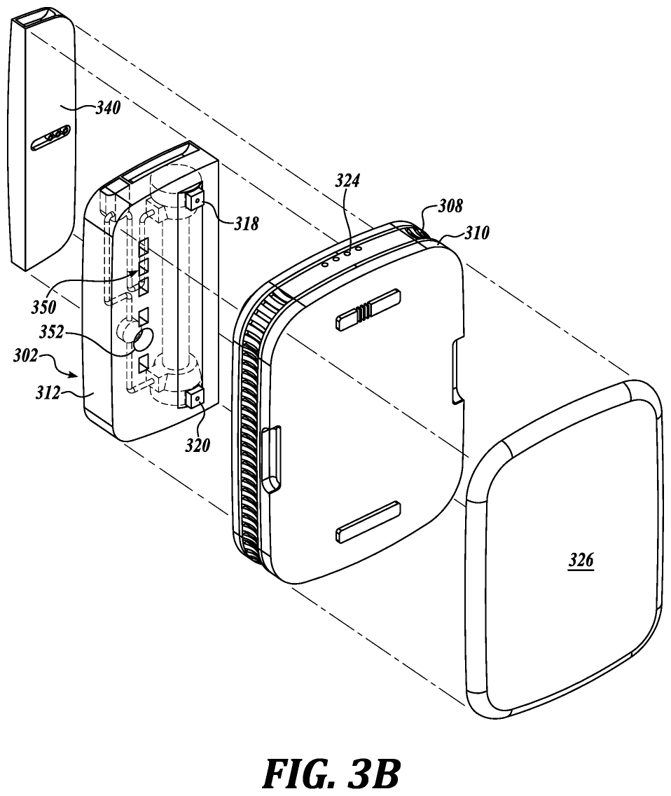

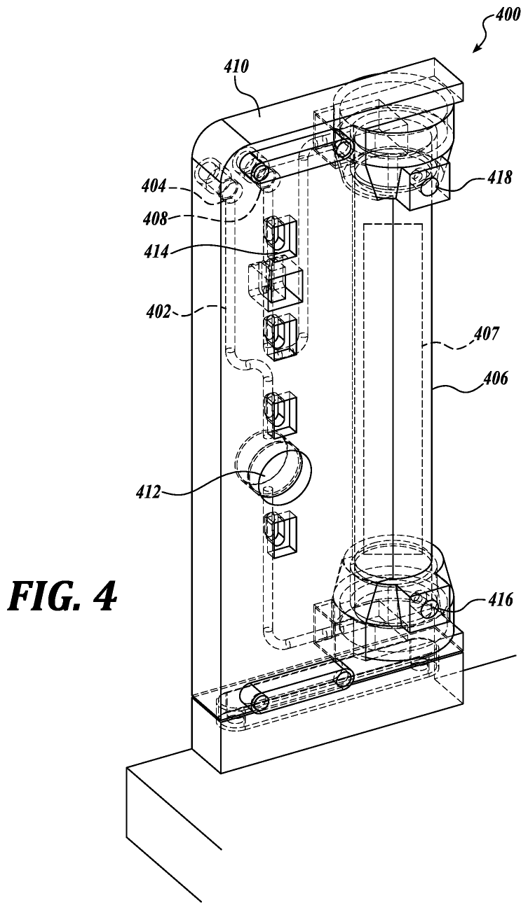

Blood handling module 104 is a consumable (disposable), modular dialyzer cartridge that enables a single patient to receive numerous dialysis treatments prior to its exhaustion. The blood handling module 104 includes a dialyzer 106, a blood conduit 108, and a portion of the dialysis fluid circuit (see FIG. 3B and FIG. 4) packaged in a blood handling module housing 110 which is configured to reversibly couple with the dialysis fluid module 102.

In an embodiment, dialyzer 106 is a hollow-fiber dialyzer having a permeable membrane of a material such as PSf (polysulfone and a family of polysulfone blends), PES (polyethersulfone), CTA (cellulose triacetate), PMMA (polymethylmethacrylate), PEPA (PES plus polyarylate), EVAL (ethylene vinyl alcohol copolymers), PAN (polyacrylonitrile), and the like. In some embodiments, a low molecular weight cut-off dialysis membrane allows only small molecules (e.g., less than 100 Da) to pass through. Suitable dialysis fluids for the hemodialysis system 100 include water-based solutions of bicarbonate and acid components.

Blood conduit 108 is a fluidic conduit (e.g., a continuous lumen) beginning with a blood inlet, passing through the dialyzer 106, and ending with a blood outlet. Each of the blood inlet and blood outlet are configured to fluidically connect with different patient blood lines, e.g., arterial or venous blood lines. In some embodiments, each of the blood inlet and blood outlet are disposed at a common location on the blood handling module 104. For example, in the illustrated embodiment, the blood inlet and blood outlet are disposed on a blood line connector 112 (e.g., a fitting, quick disconnect breakaway mechanism, or the like), configured to fluidically connect the blood inlet and blood outlet with the multi-lumen patient blood line 114.

In some embodiments, the blood line connector 112 incorporates a tension-activated quick disconnect apparatus configured to separate the blood input and the blood output from the patient blood line 114 and to seal the lumens when the quick disconnect apparatus experiences a tensile force in excess of a predetermined threshold, and optionally to stop blood flow and seal the connecting lumens to prevent blood loss when the blood line connector 112 is separated from hemodialysis system 100 in the event of an inadvertent tubing rupture.

The blood conduit 108 includes a pump interface 116 configured to operably couple with a pump disposed in the dialysis fluid module 102, such that the pump disposed in the dialysis fluid module 102 pumps blood through the blood conduit 108. For example, in some embodiments, the pump interface 116 includes a pump rotor or captive impeller which can be magnetically or mechanically driven by a pump disposed in the dialysis fluid module 102 when the blood handling module 104 is docked with the dialysis fluid module 102.

As noted above, the dialysis fluid module 102 includes a dialysis fluid conduit, which is a portion of the dialysis fluid circuit beginning with a dialysis fluid inlet, passing through the dialyzer 106 (but separated from the blood conduit 108 by the membrane), and ending with a dialysis fluid outlet (not shown in FIG. 1; see FIG. 3B). The dialysis fluid conduit of the blood handling module 104 is a segment of the larger dialysis fluid circuit of the hemodialysis system 100, which in some embodiments is a multi-pass fluid circuit and/or a closed loop circuit. Accordingly, each of the dialysis fluid inlet and the dialysis fluid outlet on the blood handling module 104 are configured to fluidically couple with a dialysis fluid outlet coupling and dialysis fluid inlet coupling, respectively, which are part of the dialysis fluid module 102.

Blood handling module housing 110 is formed from a rigid medical-grade polymer, e.g., polypropylene. In some embodiments, the module housing 110 has an overall depth which does not exceed 75 mm (e.g., about 55 mm); an overall width, measured orthogonally to the overall depth, which does not exceed 200 mm (e.g., about 155 mm); and an overall height, measured orthogonally to the overall depth and the overall width, which does not exceed 400 mm (e.g., about 310 mm). The foregoing dimensions facilitate mobility and are representative, not limiting.

The dialysis fluid module 102 is a self-contained electromechanical and/or electrochemical system that houses a system for regenerating the dialysis fluid, physical and fluidic interfaces that interface with the blood handling module 104, as well as circuitry, sensors, and pumps configured to dialyze a patient's blood by pumping it through the blood handling module 104. Accordingly, the dialysis fluid module 102 is contained in a protective housing 118 formed of at least partially of a polymer, metal, or combination thereof. In some embodiments, the housing 118 establishes the outermost physical dimensions of the hemodialysis system 100.

According to some embodiments, the hemodialysis system 100 (and in particular the housing 118) has an overall depth which does not exceed 200 mm (e.g., 150 mm); an overall width, measured orthogonally to the overall depth, which does not exceed 400 mm (e.g., 300 mm); and an overall height, measured orthogonally to the overall depth and overall width, which does not exceed 500 mm (e.g., 400 mm). In some embodiments, the hemodialysis system 100 has an overall weight that permits portability (e.g., a total weight which does not exceed 301b). These dimensions enable portability of the overall system and allow the patient to be ambulatory during dialysis.

In the embodiment shown, the dialysis fluid module 102 includes a dialysis fluid management module 120, a dialysis fluid regeneration module 122, and a fluid reservoir 124 which all fluidically couple together. Each of these sub-systems are described below.

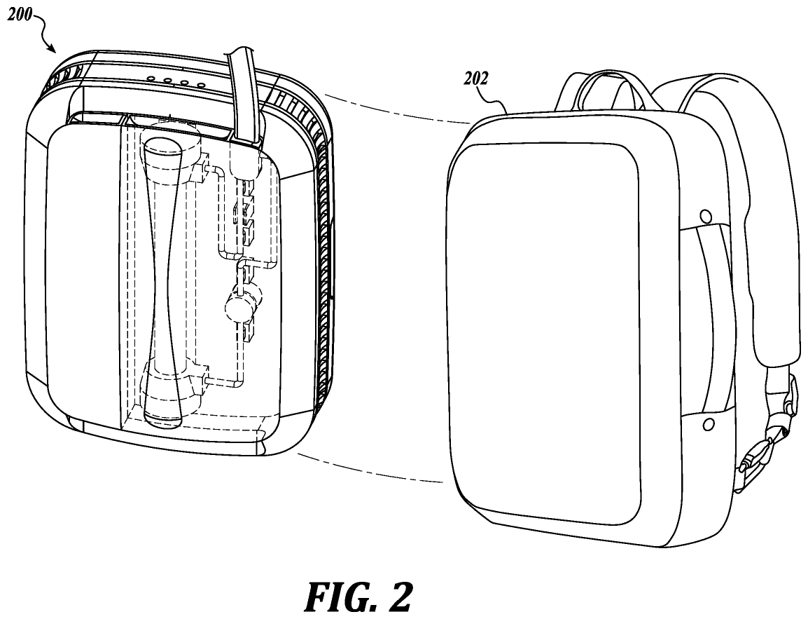

FIG. 2 shows one example of a hemodialysis system 200 configured to fit within a wearable article 202, such as a backpack, a duffel bag, a briefcase, a purse, a tote bag, a vest, a jacket, and the like. In some embodiments, the hemodialysis system 200 includes the wearable article 202.

FIG. 3A-FIG. 3B show an exploded view of a hemodialysis system 300 according to an embodiment of the present disclosure. The hemodialysis system 300 includes the features of hemodialysis system 100 described above. Accordingly, alike terms have alike meanings.

The hemodialysis system 300 includes a blood handling module 302 and a dialysis fluid module 304 disposed in a protective housing 306. Additionally, dialysis fluid module 304 includes systems to regenerate the dialysis fluid such that the hemodialysis system 300 can continuously dialyze a patient's blood with a limited dialysis fluid supply. Accordingly, the dialysis fluid module 304 of the embodiment shown includes two primary subsystems: a dialysis fluid management module 308 and a dialysis fluid regeneration module 310, described below.

The blood handling module 302 is contained within a blood handling module housing 312 that reversibly and fluidically couples with the dialysis fluid module 304. In particular, the blood handling module 302 fits at least partially within a blood handling module docking interface 314 formed at least partially as a recess in the dialysis fluid module 304. As described above, the blood handling module 302 includes a blood conduit 316 and dialysis fluid conduit. The dialysis fluid conduit extends through a dialyzer 309 and forms a portion of a larger dialysis fluid circuit which extends through both the blood handling module 302 and the dialysis fluid module 304. The blood conduit 316 also extends through the dialyzer 309.

Referring to FIG. 3B, where the blood handling module 302 docks with the docking interface 314, the dialysis fluid circuit has a dialysis fluid outlet 318 (where dialysis fluid flows out of the dialysis fluid module 304 and into the blood handling module 302) and a dialysis fluid inlet 320 (where dialysis fluid flows into the dialysis fluid module 304 from the blood handling module 302). In some embodiments, the hemodialysis system 300 has an operating orientation within +/−30 degrees from vertical, and the dialysis fluid inlet 320 is disposed gravitationally higher the dialysis fluid outlet 318 when the system 300 is in the operating orientation. This advantageously helps evacuate any gas from the blood handling module 302.

Returning to FIG. 3A, the dialysis fluid management module 308 includes an on-board control circuit 322, sensors, pumps, a user interface 324, and a portion of the dialysis fluid circuit. Additionally, dialysis fluid management module 308 provides physical, fluidic, and electrical interfaces for the blood handling module 302. These features are described below.

Control circuit 322 actuates sensors, pumps, user interface 324, and other features of the dialysis fluid module 304. A power supply 326 (e.g., a rechargeable lithium ion battery pack) is electrically connected to the control circuit 322 and powers the dialysis process. In some embodiments, the dialysis fluid management module 308 includes an AC outlet and optionally an AC/DC converter to enable connection to standardized AC power supplies, e.g., 110V, 120V, 220V, and the like.

Control circuit 322 includes a processor (e.g., a general processing unit, graphical processing unit, or application specific integrated circuit), a data store (a tangible machine-readable storage medium), a plurality of modules implemented as software logic (e.g., executable software code), firmware logic, hardware logic, or various combinations thereof.

In some embodiments, control circuit 322 includes a transceiver that transmits signals to a mobile device 328 (e.g., a smartphone, a smartwatch, a biometric sensor), and receives signals transmitted from the mobile device 328 (e.g., a signal corresponding to a sensed blood pressure), i.e., two-way communication. The data store of control circuit 322 is a tangible machine-readable storage medium that includes a mechanism that stores information in a non-transitory form accessible by a machine (e.g., the processor of control circuit 322). For example, a machine-readable storage medium includes recordable/non-recordable media (e.g., read only memory (ROM), random access memory (RAM), magnetic disk storage media, optical storage media, flash memory devices, etc.).

In some embodiments, control circuit 322 includes a communications interface having circuits configured to enable communication with the mobile device 328, and/or other network element via the internet, cellular network, RF network, Personal Area Network (PAN), Local Area Network, Wide Area Network, or other network. For example, in some embodiments, control circuit 322 transmits signals to the mobile device 328 indicative of any one or more of the parameters monitored by the sensors of the hemodialysis system 300.

Accordingly, the communications interface may be configured to communicate using wireless protocols (e.g., WIFI®, WIMAX®, BLUETOOTH®, ZIGBEE®, Cellular, Infrared, Nearfield, etc.) and/or wired protocols (Universal Serial Bus or other serial communications such as RS-216, RJ-45, etc., parallel communications bus, etc.). In some embodiments, the communications interface includes circuitry configured to initiate a discovery protocol that allows control circuit 322 and other network element to identify each other and exchange control information. In an embodiment, the communications interface has circuitry configured to a discovery protocol and to negotiate one or more pre-shared keys.

In some embodiments, the control circuit 322 is configured to pump the dialysis fluid through the dialysis fluid regeneration module 310 at a flow rate less than about 250 mL/min, 200 mL/min, 150 mL/min, 100 mL/min, or less. Such flow rates are lower than traditional hemodialysis systems, which commonly pump dialysis fluid across a dialyzer on the order of liters per minute. Further, in some embodiments, the control circuit 322 is configured to control a first pump (pumping a dialysis fluid) and the pump 334 (pumping blood through the blood handling module) to maintain a pressure gradient across a dialyzer membrane in the blood handling module 302 of between about 10 mmHg and about 300 mmHg, about 10 mmHg to about 200 mmHg, about 10 mmHg to about 100 mmHg.

When the blood handling module 302 is nested within the docking interface 314, the segment of the dialysis fluid circuit of the blood handling module 302 fluidically connects with the dialysis fluid module 304 via fluidic interfaces 330a, b (e.g., male/female fluidic couplings). In particular, dialysis fluid inlet coupling 330b provides regenerated dialysis fluid to the dialysis fluid inlet 320 of the blood handling module 302, and the fluidic dialysis fluid outlet coupling 330a receives spent dialysis fluid from the dialysis fluid outlet 318 of the blood handling module 302 (for transport back to the dialysis fluid regeneration module 310).

A blood sensor array 332 provided in the docking interface 314 is positioned such that when the blood handling module 302 is fully received within the docking interface 314, the blood sensor array 332 is positioned along the blood conduit 316 of the blood handling module 302. Accordingly, the blood sensory array 332 is configured to interface with a bloodstream (through the blood conduit 316) when the blood handling module 302 is docked with the docking interface 314.

In some embodiments, the blood sensor array 332 includes at least one of the following sensors, which are operably connected to the control circuit 322: a blood pressure sensor (which may be a single sensor or a plurality of sensors, including a sensor disposed external to the hemodialysis system 300), an oxygen level sensor, a blood flow rate sensor, a biomarker sensor (e.g., a hemoglobin sensor), a temperature sensor, a conductivity sensor, and other sensors. The foregoing sensors are non-invasive to the blood conduit of the blood handling module 302. In some embodiments in which the sensor array 332 comprises a temperature sensor and a second sensor (e.g., a conductivity sensor), the temperature sensor is configured to sense the temperature of the bloodstream at a first location along the blood conduit 316 upstream of a second location where the second sensor senses a different parameter of the blood. Advantageously, this configuration enables the control circuit 322 to make a temperature-compensated measurement with the second sensor.

In some embodiments, the sensor array 332 is configured to move between an undocked position and a docked position. In the undocked position, the sensor array 332 is not positioned to sense parameters of the bloodstream. For example, the sensor array 332 may be retracted into the housing 306, shifted to one side, or otherwise moved away from a region in the blood handling module docking interface 314 which will be occupied by the blood conduit 316 when docked. In the docked position, the sensor 332 reverts to a position where each sensor is configured to sense the corresponding parameter in the bloodstream, such as by sliding, shifting, pivoting, or extending. Advantageously, the moveable sensor array 332 facilitates docking with the blood handling module 302.

A pump 334 (such as an impeller pump, a peristaltic pump, or a linear actuator) is disposed in the docking interface 314 and operably connected to the control circuit 322 such that when the blood handling module 302 is coupled with the dialysis fluid module 304, the pump 334 pumps blood through the blood conduit 316 of the blood handling module 302. In some embodiments, pump 334 includes a pump drive disposed in the blood handling module docking interface 314, which pump drive is configured to drive (e.g., magnetically or mechanically) a captive impeller 317 disposed in the blood conduit 316 of the blood handling module 302.

Returning briefly to FIG. 3B, the blood handling module 302 includes a sensor bank interface 350 formed in the module housing 312 along the blood conduit 316, which is configured to dock with the sensor array 332. Accordingly, in some embodiments, the sensor bank interface 350 includes a plurality of recesses in the housing 312, each recess being sized, shaped, and positioned to receive a corresponding sensor of the sensor array 332 when the blood handling module 302 docks with the blood handling module docking interface 314.

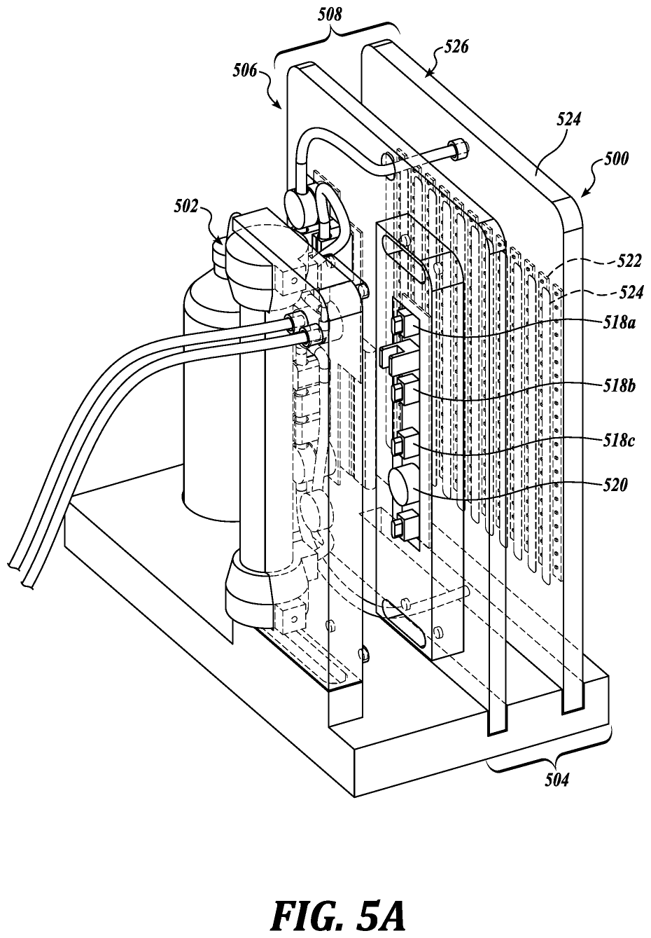

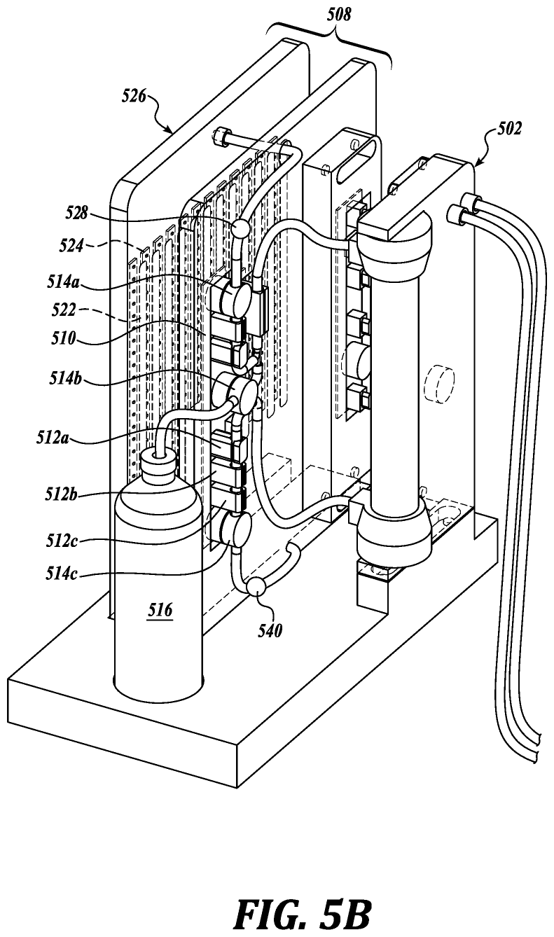

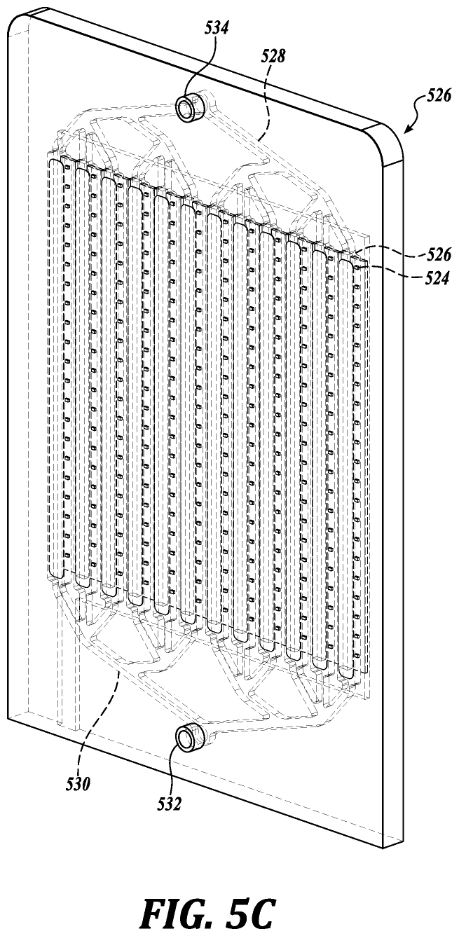

As shown in FIG. 3A and described in more detail with respect to FIG. 5B, a dialysis fluid sensor array 336 is disposed in the housing 306 and connected to the control circuit 322, and includes one or more additional sensors configured to monitor aspects of dialysis fluid circulating through the hemodialysis system 300, such as a urea level, a potassium level, and the like. Control circuit 322 is also connected to at least one dialysis fluid pump 338 disposed in the housing 306 and configured to configured to pump dialysis fluid through the dialysis fluid regeneration module 310 and optionally to pump an electrolyte solution from a fluid reservoir 340 to the dialysis fluid regeneration module 310.

A fluid reservoir 340 is a container configured to store a relatively small volume (e.g., 0.5 liters-2.0 liters) of a priming fluid, e.g., saline or water, and optionally an electrolyte solution, waste fluid, and/or excess dialysate. Accordingly, the fluid reservoir 340 comprises one or more internal containers or compartments (one for each type of fluid), each of which is fluidically coupled with the dialysis fluid circuit via a fluidic interface 342 disposed on the dialysis fluid management module 308.

Dialysis fluid regeneration module 310 is fluidically connected to the dialysis fluid circuit (which includes portions of the blood handling module 302 and dialysis fluid management module 308), and regenerates spent dialysis fluid by removing urea and other wastes dialyzed from the blood therefrom, and then returning the regenerated dialysis fluid to the blood handling module 302.

In an embodiment, the dialysis fluid regeneration module 310 incorporates a photo-oxidation process such as described in any one or more of the following: U.S. Patent Application Publication No. 20200054810, published Feb. 20, 2020; U.S. Patent Application Publication No. 20220000938, published Jan. 6, 2022; and International Patent Application No. PCT/US2019/044285, filed Jul. 31, 2019 and assigned to the University of Washington, the entirety of which are expressly incorporated herein by reference for all purposes. Such photo-oxidation processes advantageously remove urea and other wastes dialyzed from the blood and catalytically convert those wastes to carbon dioxide and nitrogen that harmlessly disperse into the atmosphere. Such photo-oxidation processes enable the hemodialysis system 300 to recirculate a relatively small volume of dialysis fluid (e.g., 0.5 liter-2.0 liters) in a closed loop system.

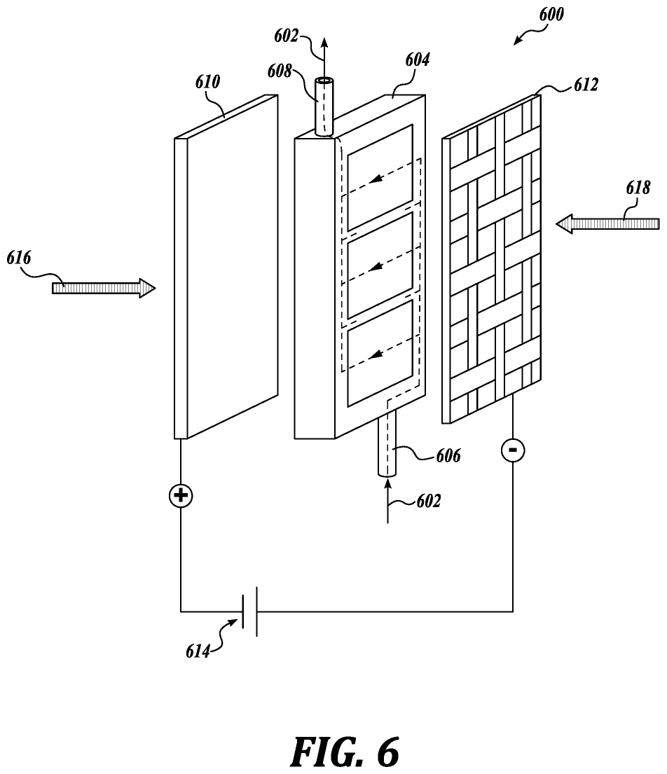

Accordingly, in some embodiments, the dialysis fluid regeneration module 310 includes at least a portion of a photo-oxidation system, for example an anode/cathode array and a light source. Representative photo-oxidation modules are described below with respect to FIGS. 5C-6.

According to other embodiments of the present disclosure, the dialysis fluid regeneration module 310 includes different systems to remove waste from the dialysis fluid, e.g., sorbent-based systems, electro-chemical oxidation systems, or other waste removal system. In sorbent-based systems, a sorbent cartridge can purify the initial dialysate and regenerate spent dialysate by passing spent dialysate through a regeneration section comprising at least one sorbent cartridge and suitable additives. The sorbent cartridges bind uremic wastes, and also can be used for other tasks, such as balancing dialysate pH. A typical sorbent cartridge system can include, for example, an activated charcoal layer (a purification layer), a urease enzyme layer (a conversion layer), a cation exchange layer, and an anion exchange layer. During regenerative dialysis, the used or spent dialysate can move up through the layers of the cartridge and a high purity regenerated dialysate can emerge from the cartridge outlet for recirculation to the dialyzer. The activated charcoal or carbon layer can be used to absorb organic metabolites such as creatinine, uric acid, and nitrogenous metabolic waste of the patient as well as chlorine and chloramines from the water. Urease used in the urease layer can be an enzyme that catalyzes the hydrolysis of urea into carbon dioxide and ammonia. Ammonium carbonate is released by a urease layer in a conventional sorbent cartridge. Ammonium created in the urease layer can be removed in the cation exchange layer, e.g., an adsorbent zirconium phosphate, in exchange for release of Na+ and H+ ions. The carbonate from the urea hydrolysis then can combine with H+ to form bicarbonate (HCO3−) and carbonic acid (H2CO3). Carbonic acid is an unstable organic acid; most of it rapidly breaks down into water and carbon dioxide molecules (CO2). The anion exchange layer, e.g., HZO containing acetate as a counter ion, can remove HCO3−, P−, and other anions (e.g., F− in water), and releases acetate.

Electro-chemical oxidation systems regenerate spent dialysis fluid by applying voltage between a cathode(s) and an anode(s) (e.g., graphite, platinum, or other suitable material) immersed in spent dialysis fluid, in order to oxidize urea and uremic toxins present in the spent dialysate fluid into nitrogen, carbon dioxide, hydrogen, and other byproducts.

Embodiments of the present disclosure which incorporate a photo-oxidation system remove both urea and non-urea toxins, and offer a number of advantages. First, as compared to sorbent-based systems, photo-oxidation systems weigh less (no sorbent is required) and avoid toxic byproducts such as ammonium. As compared to electro-chemical oxidation systems, photo-oxidation systems avoid toxic byproducts (such as chlorine), electrode leaching, and ammonium generation (which can alter blood pH levels).

In some embodiments, the hemodialysis system 300 includes a software application 344 configured for execution by the mobile device 328. Such an application includes a dialysis monitoring module that displays (on a user interface of the mobile device 328) the operational status of the 300, e.g., “dialyzing,” “priming,” “dialysis complete,” “error” based upon a signal received from the control circuit 322. In some embodiments, the application 344 includes a command module that receives an input from the user on the mobile device 328 (e.g., a “start dialysis” and/or “stop dialysis” command), and then transmits a signal from the mobile device 328 to the control circuit 322 in order to execute the command. In some embodiments, application 344 includes a dialysis operation module that senses at least one parameter with a sensor of the mobile device 328 (e.g., blood pressure), and transmits a signal corresponding to the sensed parameter to the control circuit 322, which adjusts an operational parameter of the dialysis process (e.g., a pump rate) based upon the sensed parameter. The foregoing modules are representative, not limiting, and may be executed on a plurality of mobile devices 328, e.g., a smartwatch of a patient and a smartphone of a caregiver or physician.

FIG. 4 shows a mockup of a representative blood handling module 400, revealing the functional elements thereof for greater understanding. The blood handling module 400 includes the same features as the previously introduced blood handling modules unless stated otherwise.

Blood handling module 400 includes a blood conduit 402 that receives a patient's blood via a blood inlet 404, passes through a dialyzer 406 having a dialyzer membrane 407 (as described above), and returns dialyzed blood to a blood outlet 408. In this embodiment, the blood inlet 404 and blood outlet 408 are located at a common location in order to facilitate connection to a blood line connector and/or quick disconnect apparatus. A rigid housing 410 protects the foregoing elements.

A pump head 412 is disposed in-line with the blood conduit 402, for pumping the blood from the patient and through the dialyzer 406. In an embodiment, the pump head is a rotor or impeller magnetically couplable to a pump motor disposed in the dialysis fluid module. However, some embodiments do not include such a “captive impeller” configuration. For example, embodiments utilizing a peristaltic pump may not include a captive impeller in the blood handling module.

In some embodiments, the blood handling module contains one or more sensors for monitoring and controlling the dialysis, for example a blood pressure sensor (which may be a single sensor or a plurality of sensors, including a sensor on a sensor disposed external to the hemodialysis system 300), an oxygen level sensor, a blood flow rate sensor, a biomarker sensor (e.g., a hemoglobin sensor), and/or other sensors.

A sensor interface 414 formed in the housing 410 includes a plurality of recesses, the location and size of which correspond to a blood sensor array in the dialysis fluid management module. Positioning the sensors in the dialysis fluid module rather than in the blood handling module 400 enables the sensors to be hygienically reused with different blood handling modules 400. Nevertheless, some embodiments of the blood handling module include any one or more of the sensors described above.

The blood handling module 400 incorporates a portion of the dialysis fluid circuit which passes into and out of the dialyzer 406 via dialysis fluid inlet 416 and dialysis fluid outlet 418, respectively. The portion of the dialysis fluid circuit passes along an opposite side of the dialyzer membrane 407 from the blood conduit 402.

FIG. 5A and FIG. 5B show opposite perspective views of a mockup of a representative hemodialysis system 500, according to an embodiment of the present disclosure. The hemodialysis system 500 includes the same features as previously-introduced embodiments unless stated otherwise.

As described above, the hemodialysis system 500 includes a blood handling module 502 and a dialysis fluid module 504. Any one or more of the features described with respect to the hemodialysis system 500 may be applied to any other embodiment of the present disclosure.

The blood handling module 502 has the same features as the blood handling module 400 described with respect to FIG. 4, and therefore will not be described again.

The dialysis fluid module 504 includes a dialysis fluid management module 506 fluidically connected to a dialysis fluid regeneration module 508, which together include a portion of the dialysis fluid circuit (a portion of which is also disposed in the blood handling module 502).

The dialysis fluid management module 506 includes circuitry, sensors, and pumps configured to dialyze a patient's blood by pumping it through the blood handling module 502, and also to pump dialysis fluid through the blood handling module 502 and the dialysis fluid regeneration module 508. Accordingly, dialysis fluid management module 506 includes a control circuit 510 operatively connected to a plurality of sensors, pumps, and modules which execute a number of functions which are unique to the small-volume hemodialysis systems described herein.

The control circuit 510 is operatively connected to a first plurality of sensors and pumps that pump the dialysis fluid through the system. For example, sensors 512a-c are disposed along the dialysis fluid circuit and each configured to sense a particular parameter of the dialysis fluid, e.g., a urea level, potassium level, temperature, color, or the presence and/or quantity of a gas in the dialysis fluid. Based upon one or more of the sensed parameters, the control circuit 510 may increase or decrease a flow rate of the dialysis fluid through the system, transmit a message to a user or clinician based upon the sensed parameter(s), and/or control one or more of the modules described below. For example, in some embodiments wherein the dialysis fluid regeneration module 508 comprises a photooxidation module, the control circuit 510 may increase a flow rate of the dialysis fluid through the fluid regeneration module 508 in order to increase an evaporation rate of the dialysis fluid, and therefore to remove heat from the system.

Pumps 514a-c are also disposed along the dialysis fluid circuit. In particular, pump 514a pumps regenerated dialysis fluid from the dialysis fluid regeneration module 508 to the blood handling module 502, pump 514b pumps an electrolyte solution (and/or a priming solution such as saline) from fluid reservoir 516 into the dialysis fluid circuit (e.g., to prime the circuit), and pump 514c pumps spent dialysis fluid to the dialysis fluid regeneration module 508 for regeneration. In some embodiments, pump 514b and/or another pump pumps waste fluid and/or excess dialysate fluid from the dialysis fluid circuit to the fluid reservoir 516. This is particularly advantageous if the hemodialysis system 500 removes an excess quantity of fluid off the patient during treatment. Said excess fluid can be stored in the fluid reservoir 516 (for disposal), and optionally pumped back into the dialysis fluid circuit, e.g., to regulate fluid levels and/or chemical concentrations in the system.

The control circuit 510 is also operatively connected to a second plurality of sensors and at least one pump that pumps blood through the blood handling module 502. For example, sensors 518a-c are disposed on the dialysis fluid management module 506 at a location configured to interface with the blood conduit when the blood handling module 502 is coupled to the dialysis fluid module 504. Accordingly, sensors 518a-c sense aspects of the patient's blood, e.g., a blood pressure sensor, an oxygen level sensor, a blood flow rate sensor, a biomarker sensor (e.g., a hemoglobin sensor), and the like. Pump 520 pumps blood through the blood conduit. Pump 520 may be any number of pump types. For example, in some embodiments, pump 520 is a peristaltic pump which acts on the blood conduit in the blood handling module.

In use, air or other gases may be introduced into the dialysis fluid circuit. For example, when a user primes the system with saline from the fluid reservoir 516, a small amount of air may enter the system. Or, the evaporation of dialysis fluid may create gas in the dialysis fluid circuit. Accordingly, to maintain flow rates and consistent performance within the dialysis fluid regeneration module 508, an optional gas removal module 528 is disposed along the dialysis fluid circuit and configured to remove a gas therefrom. In some embodiments, the gas removal module comprises at least one of a valve or a gas-permeable membrane in the dialysis fluid circuit, and optionally a vacuum coupled thereto in order to draw gas from the dialysis fluid circuit. To facilitate the evacuation of gas, such features may be disposed at a gravitational high point along the dialysis fluid circuit when the hemodialysis system 500 is in an upright operating orientation within +/−30 degrees from vertical.

In some embodiments, the hemodialysis system 500 is provided with an optional electrolyte management module 540 operatively connected to the control circuit 510 and in line with the dialysis fluid circuit and the fluid reservoir 516. The electrolyte management module is configured to regulate an electrolyte level in the dialysis fluid by selectively providing an electrolyte solution from the fluid reservoir 516 into the dialysis fluid circuit. Accordingly, the electrolyte management module 540 may include one or more sensors to measure an electrolyte level in the dialysis fluid, and also one or more valves disposed between the fluid reservoir 516 and the dialysis fluid circuit. In some embodiments, particularly those wherein the dialysis fluid regeneration module 518 comprises a photooxidation module, the electrolyte management module includes one or more glucose filters disposed in line with the dialysis fluid circuit at a location upstream of an inlet to the dialysis fluid regeneration module 518. Because glucose competes with urea in photooxidation processes, the glucose filter advantageously removes glucose from the dialysis fluid prior to regeneration, thereby increasing system efficiency. The foregoing sensor and pump configurations are representative, not limiting.

The dialysis fluid regeneration module 508 of the illustrated embodiment includes a photo-oxidation module which takes urea and other wastes dialyzed from the blood and catalytically converts those wastes to carbon dioxide and nitrogen that harmlessly disperse into the atmosphere.

Accordingly, in the embodiment shown in FIG. 5C, the dialysis fluid regeneration module 508 includes at least one photooxidation panel 526 formed at least partially of a translucent or transparent material (e.g., conductive FTO glass) comprising a plurality of fluidic channels 530 through which dialysis fluid flows. In particular, the fluidic channels include numerous branches stemming from a common fluid input 532, forming numerous fluidically parallel oxidation fluidic pathways 536 (where oxidation occurs), and converging to a common fluid output 534. An anode/cathode array 522 is disposed adjacent to the oxidation fluidic pathways 536 and to at least one light source 524. The anode/cathode array includes anodes and cathodes extending into lumens of the oxidation fluidic pathways 536 such that the anodes and cathodes contact the dialysis fluid flowing therethrough. In use, dialysis fluid flows through these channels under the control of the dialysis fluid management module 506.

Anode/cathode array 522 includes a plurality of anodes where oxidation occurs, e.g., TiO₂/FTO anodes hydrothermally grown on conductive FTO glass, at least one of which is disposed along each of the oxidation fluidic pathways 536 and extends into the corresponding lumens thereof. The anode/cathode array 522 also includes a plurality of cathode catalysts disposed along each of the oxidation fluidic pathways 526 and extending into the lumens thereof opposite to the corresponding anode(s), e.g., Pt/C (Pt-coated carbon paper cathodes). Thus, each oxidation fluidic pathway 526 has at least one anode and at least one cathode extending into a lumen thereof.

In some embodiments, each oxidation fluidic pathway 526 includes a single elongate anode and a single elongate cathode extending along a length thereof; however, other embodiments include a plurality of discrete anodes and cathodes extending along each oxidation fluidic pathway 536. According to some embodiments, the anode/cathode array(s) 522 have a combined active surface area (exposed to the lumen of the oxidation fluidic pathway(s) 536) of at least approximately 0.25-2.0 square feet, in order to remove at least 12-15 g of urea in 24 hours (a representative level of human urea production).

Light source 524 includes an LED array, UV lamps, or other light source that faces the anodes of the anode/cathode array 522 and provides photo-activation energy to initiate urea oxidation. In the illustrated embodiment, a plurality of LEDs are disposed along each oxidation fluidic pathway 536. Nevertheless, some embodiments may utilize a centralized light source 524 that provides light to a plurality of fluidic oxidation pathways 536. Although LEDs are used in the illustrated light source 524 for their efficiency and output, other embodiments may include other forms of illumination.

The illustrated embodiment includes a single photooxidation panel 526. Nevertheless, some embodiments include a plurality of photooxidation panels 526, e.g., 2, 3, 4, 5, 6, 7, 8, 9, or 10 such arrays, which may be fluidically connected in parallel (to increase urea removal capacity) and/or in series (to provide additional urea removal stages). Some embodiments utilize a plurality of light sources 524, for example a plurality of light panels in connection with a plurality of anode/cathode arrays 522; in such embodiments, a single light source 524 may be configured to illuminate more than one anode/cathode array 522.

Thus, according to a representative process of the present disclosure, spent dialysis fluid is pumped into the panel 526 at fluid input 532 under control of the dialysis fluid management module 506, photo-oxidized in the oxidation fluidic pathways 536 by the light source(s) 524 and anode/cathode array(s) 522, causing urea in the spent dialysis fluid to decompose into CO₂, N₂ and H₂O. The water component of the regenerated dialysate exits the panel 526 at fluid output 534 and is recirculated by the pump 514a into the dialysis fluid circuit for reuse.

In some embodiments, the dialysis fluid regeneration module 508 includes a different anode/cathode array than that shown in the illustrated embodiment. As one example, the anode/cathode array is formed as a flexible panel of cells (e.g., circular cells) connected in parallel, each cell having a disk with an anode side, a cathode side, and a light source therein. Such a flexible panel advantageously enables conformity to a wearer's body, while the parallel fluidic connection increases toxin removal efficiency. The foregoing examples are representative, not limiting.

Testing of a representative hemodialysis system of the present disclosure resulted in the successful removal of an average of 14.0±0.2 grams of urea from a patient solution stream (a solution of saline and urea at physiological relevant concentration, formulated to mimic a patient's blood) over 24 hours of continuous operation, utilizing only regenerated dialysis fluid.

FIG. 6A is an exploded view of another representative photo-oxidation module 600 which may be embodied in any dialysis fluid regeneration module of the present disclosure. The process described below is representative of the process which occurs in the photo-oxidation module described with respect to FIGS. 5A-5C.

The electrochemical reaction that takes place in the photo-oxidation module 600 may be described as:

Anode: CO(NH₂)₂+6OH⁻→CO₂+N₂+5H₂O+6e⁻

Cathode: O₂+2H₂O+4e⁻→4OH⁻

Net: CO(NH₂)₂+3/2O₂→CO₂+N₂+2H₂O (Eq. 1)

In some embodiments, dialysis fluid 602 flows through a spacer 604 from an inlet 606 to an outlet 608. Dialysis fluid 602 carries urea that is to be electrochemically decomposed into CO₂ and N₂. The spacer 604 may be sandwiched between an anode 610 and a cathode 612, each individually connected to a source of voltage 614 (e.g., a battery or other power source providing DC voltage). In some embodiments the source of voltage 614 provides voltage differential within a range from about 0.6 V to about 0.8 V. In some embodiments of spacer 604, the entire dialysate flow is directed to flow over a TiO₂ layer.

In some embodiments, the anode 610 is fitted with nanostructures (e.g., TiO₂ nanowires). In operation, the anode 610 is illuminated by a source of light 616 that emits light (e.g., UV light) for the electrochemical reaction shown in equation 1. At the anode 610, photo-excited TiO₂ nanostructures provide holes for the oxidation of solution species on the surface, while electrons are collected on underlying conducting oxide (e.g., fluorine doped thin oxide or FTO), and then transported to the cathode electrode to split water into OH⁻. The photo-excitation may be provided by the source of light 616 or by natural light.

In some embodiments, the cathode 612 may be gas permeable (e.g., air permeable or oxygen permeable). In operation, a flow of gas 618 that includes oxygen can pass through the cathode 612 toward the dialysate that includes urea.

In some embodiments, the photo-oxidation module may be used for preparing or regenerating a dialysis fluid. For example, water to be treated may be passed between the anode 610 and the cathode 612 to oxidize impurities in the water to be treated, thereby generating the dialysis fluid.

The foregoing photo-oxidation module 600 is representative, not limiting, of photo-oxidation modules which may be embodied in dialysis fluid regeneration modules of the present disclosure.

The present application may also reference quantities and numbers. Unless specifically stated, such quantities and numbers are not to be considered restrictive, but representative of the possible quantities or numbers associated with the present application. Also in this regard, the present application may use the term “plurality” to reference a quantity or number. In this regard, the term “plurality” is meant to be any number that is more than one, for example, two, three, four, five, etc. The terms “about,” “approximately,” “near,” etc., mean plus or minus 5% of the stated value. For the purposes of the present disclosure, the phrase “at least one of A, B, and C,” for example, means (A), (B), (C), (A and B), (A and C), (B and C), or (A, B, and C), including all further possible permutations when greater than three elements are listed.

Embodiments disclosed herein may utilize circuitry in order to implement technologies and methodologies described herein, operatively connect two or more components, generate information, determine operation conditions, control an appliance, device, or method, and/or the like. Circuitry of any type can be used. In an embodiment, circuitry includes, among other things, one or more computing devices such as a processor (e.g., a microprocessor), a central processing unit (CPU), a digital signal processor (DSP), an application-specific integrated circuit (ASIC), a field-programmable gate array (FPGA), or the like, or any combinations thereof, and can include discrete digital or analog circuit elements or electronics, or combinations thereof.

In an embodiment, circuitry includes one or more ASICs having a plurality of predefined logic components. In an embodiment, circuitry includes one or more FPGA having a plurality of programmable logic components. In an embodiment, circuitry includes hardware circuit implementations (e.g., implementations in analog circuitry, implementations in digital circuitry, and the like, and combinations thereof). In an embodiment, circuitry includes combinations of circuits and computer program products having software or firmware instructions stored on one or more computer readable memories that work together to cause a device to perform one or more methodologies or technologies described herein. In an embodiment, circuitry includes circuits, such as, for example, microprocessors or portions of microprocessor, that require software, firmware, and the like for operation. In an embodiment, circuitry includes an implementation comprising one or more processors or portions thereof and accompanying software, firmware, hardware, and the like. In an embodiment, circuitry includes a baseband integrated circuit or applications processor integrated circuit or a similar integrated circuit in a server, a cellular network device, other network device, or other computing device. In an embodiment, circuitry includes one or more remotely located components. In an embodiment, remotely located components are operatively connected via wireless communication. In an embodiment, remotely located components are operatively connected via one or more receivers, transmitters, transceivers, or the like.

An embodiment includes one or more data stores that, for example, store instructions or data. Non-limiting examples of one or more data stores include volatile memory (e.g., Random Access memory (RAM), Dynamic Random Access memory (DRAM), or the like), non-volatile memory (e.g., Read-Only memory (ROM), Electrically Erasable Programmable Read-Only memory (EEPROM), Compact Disc Read-Only memory (CD-ROM), or the like), persistent memory, or the like. Further non-limiting examples of one or more data stores include Erasable Programmable Read-Only memory (EPROM), flash memory, or the like. The one or more data stores can be connected to, for example, one or more computing devices by one or more instructions, data, or power buses.

In an embodiment, circuitry includes one or more computer-readable media drives, interface sockets, Universal Serial Bus (USB) ports, memory card slots, or the like, and one or more input/output components such as, for example, a graphical user interface, a display, a keyboard, a keypad, a trackball, a joystick, a touch-screen, a mouse, a switch, a dial, or the like, and any other peripheral device. In an embodiment, circuitry includes one or more user input/output components that are operatively connected to at least one computing device to control (electrical, electromechanical, software-implemented, firmware-implemented, or other control, or combinations thereof) one or more aspects of the embodiment.

In an embodiment, circuitry includes a computer-readable media drive or memory slot configured to accept signal-bearing medium (e.g., computer-readable memory media, computer-readable recording media, or the like). In an embodiment, a program for causing a system to execute any of the disclosed methods can be stored on, for example, a computer-readable recording medium (CRMM), a signal-bearing medium, or the like. Non-limiting examples of signal-bearing media include a recordable type medium such as any form of flash memory, magnetic tape, floppy disk, a hard disk drive, a Compact Disc (CD), a Digital Video Disk (DVD), Blu-Ray Disc, a digital tape, a computer memory, or the like, as well as transmission type medium such as a digital and/or an analog communication medium (e.g., a fiber optic cable, a waveguide, a wired communications link, a wireless communication link (e.g., transmitter, receiver, transceiver, transmission logic, reception logic, etc.). Further non-limiting examples of signal-bearing media include, but are not limited to, DVD-ROM, DVD-RAM, DVD+RW, DVD-RW, DVD-R, DVD+R, CD-ROM, Super Audio CD, CD-R, CD+R, CD+RW, CD-RW, Video Compact Discs, Super Video Discs, flash memory, magnetic tape, magneto-optic disk, MINIDISC, non-volatile memory card, EEPROM, optical disk, optical storage, RAM, ROM, system memory, web server, or the like.

The detailed description set forth above in connection with the appended drawings, where like numerals reference like elements, are intended as a description of various embodiments of the present disclosure and are not intended to represent the only embodiments. Each embodiment described in this disclosure is provided merely as an example or illustration and should not be construed as preferred or advantageous over other embodiments. The illustrative examples provided herein are not intended to be exhaustive or to limit the disclosure to the precise forms disclosed. Similarly, any steps described herein may be interchangeable with other steps, or combinations of steps, in order to achieve the same or substantially similar result. Generally, the embodiments disclosed herein are non-limiting, and the inventors contemplate that other embodiments within the scope of this disclosure may include structures and functionalities from more than one specific embodiment shown in the figures and described in the specification.

In the foregoing description, specific details are set forth to provide a thorough understanding of exemplary embodiments of the present disclosure. It will be apparent to one skilled in the art, however, that the embodiments disclosed herein may be practiced without embodying all the specific details. In some instances, well-known process steps have not been described in detail in order not to unnecessarily obscure various aspects of the present disclosure. Further, it will be appreciated that additional embodiments of the present disclosure may employ any combination of features described herein.

The present application may include references to directions, such as “vertical,” “horizontal,” “front,” “rear,” “left,” “right,” “top,” and “bottom,” etc. These references, and other similar references in the present application, are intended to assist in helping describe and understand the particular embodiment (such as when the embodiment is positioned for use) and are not intended to limit the present disclosure to these directions or locations.

The present application may also reference quantities and numbers. Unless specifically stated, such quantities and numbers are not to be considered restrictive, but exemplary of the possible quantities or numbers associated with the present application. Also in this regard, the present application may use the term “plurality” to reference a quantity or number. In this regard, the term “plurality” is meant to be any number that is more than one, for example, two, three, four, five, etc. The term “about,” “approximately,” etc., means plus or minus 5% of the stated value. The term “based upon” means “based at least partially upon.”

The principles, representative embodiments, and modes of operation of the present disclosure have been described in the foregoing description. However, aspects of the present disclosure, which are intended to be protected, are not to be construed as limited to the particular embodiments disclosed. Further, the embodiments described herein are to be regarded as illustrative rather than restrictive. It will be appreciated that variations and changes may be made by others, and equivalents employed, without departing from the spirit of the present disclosure. Accordingly, it is expressly intended that all such variations, changes, and equivalents fall within the spirit and scope of the present disclosure as claimed.

1. A portable hemodialysis system, comprising:
a housing at least partially storing a dialysis fluid regeneration module configured to remove urea from a dialysis fluid, and a dialysis fluid management module comprising a control circuit operably connected to a first pump fluidically connected with the dialysis fluid regeneration module, and
a blood handling module fluidically connectable to the dialysis fluid regeneration module at a blood handling module docking interface formed in the housing, wherein the blood handling module comprises a blood conduit and a dialysis fluid conduit extending along a dialyzer membrane,
wherein the dialysis fluid management module is configured to actuate the first pump to pump the dialysis fluid through the dialysis fluid regeneration module and through the dialysis fluid conduit of the blood handling module when the blood handling module is docked at the blood handling module docking interface.

2. The portable hemodialysis system of claim 1, wherein the dialysis fluid regeneration module comprises at least one of a photo-oxidation module, an electro-oxidation module, or a sorbent module.

3. The portable hemodialysis system of claim 2, wherein the dialysis fluid regeneration module comprises the photo-oxidation module, and wherein the photo-oxidation module comprises an anode/cathode array disposed adjacent to a light source.

4. The portable hemodialysis system of claim 3, wherein the anode/cathode array is formed as a panel comprising a plurality of dialysate fluid channels formed therein.

5. The portable hemodialysis system of claim 1, wherein the control circuit is configured to pump the dialysis fluid through the dialysis fluid regeneration module at a flow rate less than about 200 mL/min.

6. The portable hemodialysis system of claim 5, wherein the control circuit is operably connected to a second pump disposed in the housing and configured to pump a bloodstream through the blood conduit of the blood handling module docked with the blood handling module docking interface, wherein the control circuit is configured to control the first pump and the second pump to maintain a pressure gradient across the dialyzer membrane of between about 10 mmHg and about 300 mmHg.

7. The portable hemodialysis system of claim 1, wherein the dialysis fluid regeneration module is fluidically connected to a gas removal module configured to remove a gas from the dialysis fluid through at least one of a valve or a gas-permeable membrane.

8. The portable hemodialysis system of claim 7, wherein the gas removal module comprises a vacuum configured to draw gas from the dialysis fluid.

9. The portable hemodialysis system of claim 1, wherein the control circuit regulates evaporation of the dialysis fluid by modulating a urea removal rate in the dialysis fluid regeneration module, based upon at least one of a temperature of the dialysis fluid or a volume of the dialysis fluid.

10. The portable hemodialysis system of claim 1, wherein the dialysis fluid conduit of the blood handling module receives the dialysis fluid from a dialysis fluid outlet coupling and provides the dialysis fluid to a dialysis fluid inlet coupling formed in the blood handling module docking interface, the dialysis fluid outlet coupling and the dialysis fluid inlet coupling being in fluidic connection with the dialysis fluid regeneration module.

11. The portable hemodialysis system of claim 10, wherein the portable hemodialysis system has an operating orientation wherein the dialysis fluid inlet coupling is disposed gravitationally higher the dialysis fluid outlet coupling.

12. The portable hemodialysis system of claim 1, wherein the blood handling module docking interface comprises a sensor array configured to interface with a bloodstream in the blood conduit when the blood handling module is docked with the blood handling module docking interface.

13. The portable hemodialysis system of claim 12, wherein the sensor array comprises a temperature sensor and a second sensor respectively configured to measure a temperature and a second parameter of the bloodstream, wherein the temperature sensor is configured to sense the temperature of the bloodstream at a first location in the bloodstream upstream of a second location in the bloodstream where the second sensor senses the second parameter of the bloodstream.

14. The portable hemodialysis system of claim 13, wherein the control circuit is configured to cause the sensor array to move between an undocked position and a docked position.

15. The portable hemodialysis system of claim 1, further comprising a fluid reservoir reversibly coupled with a reservoir docking interface of the housing, wherein the fluid reservoir is in fluidic connection with the dialysis fluid regeneration module.

16. The portable hemodialysis system of claim 15, wherein the control circuit is operatively connected to an electrolyte management module configured to regulate an electrolyte level in the dialysis fluid by providing electrolytes from the fluid reservoir.

17. The portable hemodialysis system of claim 15, wherein the fluid reservoir comprises at least one of an electrolyte solution container, a saline reservoir, a waste fluid reservoir, or an excess dialysate fluid reservoir.

18. The portable hemodialysis system of any claim 17, further comprising a second pump fluidically connected to the fluid reservoir and the dialysis fluid regeneration module.

19. The portable hemodialysis system of claim 1, further comprising a power supply operably connected to the control circuit.

20. The portable hemodialysis system of claim 1, further comprising a wearable article containing the dialysis fluid regeneration module and the blood handling module, the wearable article being selected from the group consisting of: a backpack, a vest, a bag, a briefcase, a purse, and a jacket.

21. A portable hemodialysis system, comprising:
a housing at least partially storing a dialysis fluid regeneration module configured to remove urea from a dialysis fluid, and a dialysis fluid management module comprising a control circuit operably connected to a first pump fluidically coupled to pump the dialysis fluid through the dialysis fluid regeneration module,
wherein a blood handling module docking interface is formed in the housing, comprising a dialysis fluid inlet coupling and a dialysis fluid outlet coupling in fluidic connection with the dialysis fluid regeneration module, and at least a portion of a second pump configured to pump blood through a blood conduit of a blood handling module docked with the blood handling module docking interface, wherein the blood handling module comprises the blood conduit and a dialysis fluid conduit extending along a dialyzer membrane, the dialysis fluid conduit extending from the dialysis fluid inlet coupling to the dialysis fluid outlet coupling when the blood handling module is docked with the blood handling module docking interface.

22. A blood handling module, comprising: a housing storing a blood conduit and a dialysis fluid conduit extending along opposite sides of a dialyzer membrane, wherein the blood conduit comprises a blood input and a blood output extending through the housing, wherein the dialysis fluid conduit comprises a dialysis fluid inlet and a dialysis fluid outlet extending through the housing, wherein in an operating position of the blood handling module, the dialysis fluid outlet is disposed gravitationally higher than the dialysis fluid inlet, and wherein a pump interface is disposed along the blood conduit, the pump interface being configured to operably couple with an external pump.

23-42. (canceled)
